(19) United States
(12) Patent Application Publication
(10) Pub. No.: US 2024/0127968 A1
(43) Pub. Date: Apr. 18, 2024

(54) LEARNING FROM TRIAGE ANNOTATIONS

(71) Applicant: BenevolentAI Technology Limited, London (GB)

(72) Inventors: Daniel Lawrence NEIL, Williamsburg, VA (US); Dane Sterling CORNEIL, Brooklyn, NY (US); Vinay Prashanth SUBBIAH, Brooklyn, NY (US); Rachel HODOS, Staten Island, NY (US)

(73) Assignee: BenevolentAI Technology Limited, London (GB)

(21) Appl. No.: 18/491,988

(22) Filed: Oct. 23, 2023

Related U.S. Application Data
(63) Continuation of application No. PCT/EP22/60781, filed on Apr. 22, 2022.
(60) Provisional application No. 63/178,052, filed on Apr. 22, 2021.

(51) Int. Cl. G16H 70/40; G06N 20/00

(52) U.S. Cl. CPC G16H 70/40; G06N 20/00

(57) ABSTRACT

Herein disclosed are a methods and systems of SAMMI—a machine learning-based workflow that uses human annotations as labels for training models—used to predict human-based annotations for drug discovery. SAMMI receives an input to a model trained using human-annotated data, wherein the human-annotated data comprises at least one annotation associated with a triage-progressability annotation of whether to progress the input for the drug discovery. SAMMI also receives a set of features. The set of features are associated with the input, the model, and the triage-progressability of the input. The set of features is applied to the model to predict whether the input is triage-progressible. A model output is provided based on the prediction.

### CROSS-REFERENCE TO RELATED APPLICATIONS

The present application is a bypass continuation of International Application No. PCT/EP2022/060781, filed Apr. 22, 2022, which in turn claims the priority benefit, under 35 U.S.C. 119(e), of U.S. Application No. 63/178,052, filed Apr. 22, 2021. Each of these applications is incorporated herein by reference in its entirety for all purposes.

### FIELD

The present application relates to a system, apparatus and method(s) for assessing human-based annotations to improve the results of drug discovery in silico using triage progressability described herein.

### BACKGROUND

In drug discovery, a particular target must first meet specific requirements before considered as a candidate for further development and commercialization. For instance, the target should have a promising toxicity profile with the desired biological activity and suitable expression level in disease-affected tissues. The target should also be novel or may be a part of a relevant biological pathway. The data collected as part of these requirements will be crucial in providing criteria or baseline standards that drug discovery experts could utilize, whether it is to develop a more meaningful target prioritization and for identifying new drugs.

Often, these criteria are not taken into account at various stages of drug discovery, especially in the context of deploying machine learning (ML) models trained based on the criteria. Many of the existing deployed models focus on the biological rationale of the target while neglecting other criteria. Models that do consider these criteria still fail to capture whether the target is in the “sweet spot” of evidence that makes it progressible (i.e. disease-relevant, but novel enough to be promising for new commercial development), progressing to the next development stage. Thus, greater than 60% of targets continue to be rejected post-progression as these criteria are evidently not considered by the models.

A majority of the existing references describe ML models for learning human annotations or labels in the context of drug discovery. These references include target prediction tools such as TargetDB (an aggregation tool for public target data that includes a machine learning classifier for tractability). However, most of these target prediction tools take little or no account of human experts' decision-making process or the understanding of how drug discovery experts make decisions and the breadth of factors that must be taken into account to reliably determine drug target candidates suitable for progressing through drug discovery. For example, in the case of TargetDB, it does not describe whether the target annotations are made to a particular disease of interest or consider how much the annotator is concerned with factors such as safety, ligandability, and disease-respective novelty. Overall, it is apparent that target prediction tools in the public domain are not effective in reliably predicting suitable drug targets.

Consequently, there is a need for an improved computer implemented method of identifying suitable drug target candidates for drug discovery. In particular there is a need for a method in which a broader range of requirements are taken into account in determining the suitability of a target. Given presently, a significant proportion of target identification is carried out manually, requiring significant input of an experienced drug discovery expert, there is also a need for a method which can automate this process to more efficiently and reliably identify targets, to expedite the process of drug discovery. To address this need or any problem outlined in this application, at least in part, SAMMI—a machine learning-based workflow that uses human annotations as labels for training models—has been developed and described herein as a method, system, medium and/or apparatus.

It is further understood that the embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides the methods and systems of applying a machine learning-based workflow that uses human annotations as labels for training models to identify suitable drug targets, or “SAMMI”. SAMMI provides a prediction of whether an input drug target is “triage progressible”, that is, whether it suitable to form the basis of further drug discovery work and therefore helps human experts evaluate whether to progress an input as a potential drug target candidate based on an indication of human-based annotation. The model underlying SAMMI is trained using human-annotated data. In particular, the model is trained using labelled drug target data where a human expert has labelled the drug target, based on a number of criteria such as a disease of interest and their experience in the field, as being suitable to form the basis of further drug discovery work. The training data therefor comprises a “human-based” annotation indicating whether the drug target is triage progressible based on a number of criteria and the model is trained to predict the label based on features encoding information associated with the input drug target candidate and the criteria.

During evaluation of an unseen drug target candidate, the trained model is applied to a new input drug target and outputs a prediction of a “human based” annotation indicating whether the input drug target is triage progressible, i.e. whether it meets the requirements of a suitable drug target for onward drug discovery development work. Features encoding input information relating to the drug target and the criteria (such as a disease of interest, the required safety threshold and the required novelty threshold) are input into the trained model and the model provides an output prediction of whether a human expert would label the target as triage progressible under the specified input criteria.

In a first aspect, the present disclosure provides a computer-implemented method of predicting human-based annotations for drug discovery, the method comprising: receiving an input to a model trained using human-annotated data, wherein the human-annotated data comprises at least one annotation associated with a triage-progressability annotation of whether to progress the input for the drug discovery; receiving a set of features associated with the input, the model, and the triage-progressability of the input; applying the set of features to the model to predict whether the input is triage-progressible; and providing a model output based on the prediction.

The phrase “human-based annotations” refers to annotations provided to training data indicating whether a particular target is triage progressible based on specified input criteria. The annotation may be a label indicating the triage-progressibility of the input. For example, the annotation may be a binary value indicating whether or not the drug target is deemed triage progressible by a human expert. In some examples the annotation may be a non-binary score indicating the likelihood of success of the target. The phrase “triage-progressible” refers to a target which is deemed suitable for drug discovery, i.e. a target which is deemed to meet the requirements to be progressed, such as toxicity, therapeutic evidence, novelty and ligandability.

The set of features preferably encode relevant information from the input usable by the model to provide the prediction. For example the features comprise target metadata, where target metadata is data describing the target. The input preferably further comprises one or more user specified criteria, such as a target disease. The user specified criteria may also comprise, for example, a biological mechanism of interest, the relative importance of safety and/or novelty. In this case the set of features further encode the user specified criteria, together with the input target, and are input into the trained model to provide the output prediction of the triage progressibility of the target under the specified criteria.

Alternatively stated, the first aspect of the invention provides a computer-implemented method of evaluating a drug target candidate for drug discovery, the method comprising: receiving an input indicating a drug target candidate to be evaluated; determining a set of features associated with the input; inputting the features into a triage-progressibility model, the triage progressibility model comprising a machine learning model trained to predict, based on the set of features, an annotation indicating whether or not the drug target candidate is triage progressible; providing a model output based on the prediction. The annotation comprises a label representing a human assessment of the predicted success of the input target for future drug discovery. Alternatively stated, the model is trained to provide a prediction of a human expert's assessment of the likelihood of success of the drug target candidate as the basis of future drug discovery, i.e. whether it is “triage progressible”.

The present invention allows for the model to take into account a wider range of input factors than existing methods, which generally apply machine learning to focus on a narrower range of features based on pure biological rationale. In this way, more effective drug target candidates can be identified, more reliably, with reduced human input significantly increasing the efficiency of the process of evaluating drug targets for discovery. Notably, the invention departs from the generally accepted principle in the field of machine learning for drug discovery that models should be trained to process training data to spot patterns that human experts might miss to identify new candidates. In this case, the model is trained to mimic human decision making, by training the model to take into account a broader range of factors in predicting a human assessment of the potential of one or more drug target candidates.

The step of determining a set of features preferably comprises computing or extracting a set of data features encoding information associated with the input. The set of features preferably comprises target metadata encoding relevant information associated with the target such as the target family. The set of features are associated with the input in that they encode relevant information associated with the input, usable by the model to provide the output prediction. In this way the set of features may be defined as “associated with the input, the model, and the triage-progressability of the input”.

The annotation may be defined as a “human-based annotation”, meaning that the model is trained on annotated training data which has been annotated by a human with a label indicating whether they consider the target suitable for drug discovery, i.e. whether it is “triage progressible”. The trained model then provides as an output a prediction of the human-based annotation, when the features encoding the input are input into (or equivalently “applied to”) the trained model.

The model output based on the prediction may be the predicted annotation indicating whether the drug target candidate is triage progressible, e.g. a binary value (i.e. triage progessible/not triage progressible, or equivalently “MATCH” or “NOT MATCH”, as used herein). The model output may alternatively or additionally comprise a value, for example a decimal between 0 and 1, indicating a likelihood of the input being triage progressible or not, for example a regression output or confidence level in the prediction.

Preferably the annotation comprises a binary label representing a human assessment of whether or not the drug target candidate should be progressed for drug discovery. This binary label is applied by a human expert to the training data used to train the model. The trained model then provides a prediction of the binary label. In other examples, the output may comprise a number of predictions for example whether the model predicts that the target meets one or more of the user specified criteria, such as a safety threshold, novelty threshold, biological rationale threshold and/or therapeutic evidence threshold. These “sub predictions” may be combined, for example based on a specified or model-determined weighting, to provide the overall prediction of triage progressibility.

Preferably the input further comprises an indication of a disease of interest for which the drug target is to be evaluated and the set of features comprise features encoding disease and target metadata, where the machine learning model is trained to predict, based on the set of features, a human annotation indicating whether or not the drug target candidate should be progressed for drug discovery for the disease of interest. In this way the model determines a likelihood of success of the input disease target for a selected disease of interest, where the evaluation metric may differ, providing a more reliable indication of the triage-progressibility of the disease target for the intended disease. The features may comprise disease-target features such as one or more of: the output score of a biological performance model; the number of clinical trials that have been carried out; an assessment of degree of association between the target and disease determined from analysis of the literature.

Preferably the input further comprises one of more user specified criteria and the set of features comprise features encoding the user specified criteria, where the user-specified criteria comprising one or more of: a safety threshold; a therapeutic evidence threshold; a novelty threshold; a disease of interest; a biological mechanism of interest; a ligandibility threshold. When the training data is annotated by a human expert, the human expert provides the annotation indicating triage progressibility in relation to one or more of the user specified criteria, for example the human expert may indicates that a target is progressible based on a required safety threshold and novelty threshold (i.e. based on the number of published studies on the target). During training, the user specified criteria are provided to the model and the model is trained to predict the human annotated label based on the specified criteria and other input features. In this way, when applying the trained model to assess a target, the user may select one or more criteria which are provided as inputs to the trained model. The model then provides an output prediction of the progressibility (e.g. a binary yes/no or MATCH/NOT MATCH or a progressbility score) based on the input criteria. In this way, the invention can take into account a wider range of factors in assessing the triage progessibility of the selected target.

The set of features may comprise a score output by a biological performance model for the input target. For example, a separate model trained to provide a predicted biological performance score for a selected target and disease may be used to provide an input feature into the triage-progressibility model. This is an example of an “indirectly informative feature” described below, that is not seen by the human expert when labelling the training data but is nevertheless predictive of their assessment.

Preferably the triage-progressibility model is a machine learning model trained by: providing a labelled data set comprising data describing a plurality of drug targets, where each drug target has been manually labelled with a binary label indicating a positive or negative human assessment of whether the drug target is deemed suitable for progression based on one or more criteria; determining a set of features associated with each drug target and the corresponding criteria; training the model to predict the binary label based on the set of features. The criteria may comprise one or more of: a safety threshold; a therapeutic evidence threshold; a novelty threshold; a disease of interest; a biological mechanism of interest; a ligandibility threshold. The features encoding the thresholds are preferably a number representing the threshold, for example “deployment_safety_importance: 2” and deployment_ligandability_importance: 3″

The machine learning model preferably comprises a classifier or regression model. For example the machine learning model may comprise a model based on decision trees which has been determined to provide a particularly processing efficient architecture to achieve reliable results. More specifically, the model may be based on gradient boosted decision trees.

In a second aspect, the present disclosure provides a computer-implemented method for providing a model for predicting triage-progressability, wherein the method comprising: receiving a subset of human-annotated data, wherein the subset of human-annotated data is annotated for triage-progressability; identifying a set of model features for the subset of human-annotated data; classifying the subset of human-annotated data based on the set of model features; and updating the model to evaluate whether the set of human-annotated data is triage-progressible, wherein the model is configured to generate the triage-progressability associated with a model output, wherein the model is used for evaluation of whether a set of data is triage-progressible.

In a third aspect, the present disclosure provides a computer-implemented method for ranking drug targets based on triage-progressability of the drug targets, wherein the method comprising: receiving a target-specified dataset and a set of targets, wherein the target-specified dataset comprises one or more of: a set of model, literature source, and alternative source of data; scoring the set of targets using the target-specified dataset based on biological relevance; aggregating said scoring to provide a ranked list of aggregate scores, wherein the ranked list comprises a list of targets and corresponding predictions ranked according to said scoring; providing the ranked list to a model for prediciting triage-progressability, wherein the model is configured to predict a triage-progressability score for each target of the ranked list; and ranking the set of targets based on the triage-progressability score predicted by assessing the triage-progressability.

In a fourth aspect, the present disclosure provides a system for predicting human-based annotations for drug discovery, wherein the system further comprising: an input module configured to receive an input to a model trained using human-annotated data, wherein the human-annotated data comprises at least one annotation associated with a triage-progressability of whether to progress the input for the drug discovery; the input module is further configured to receive a set of features associated with the input, the model, and the triage-progressability of the input; an evaluation module configured to apply the set of features to the model, predicting whether the input is progressible; an output module configured to provide a model output based on the prediction.

In a fifth aspect, the present disclosure provides a system for drug discovery based on triage-progressability, wherein the system is configured to: receive a target-specified dataset and a set of targets, wherein the target-specified dataset comprises one or more of: a set of models, a collection of literature sources, and a compilation of data from external sources; rank the set of targets based on the target-specified dataset to generate a ranked list; provide the ranked list to a model for predicting the triage-progressability, wherein the model is configured to predict a triage-progressability score for each target of the ranked list; and for each target of the ranked list, the model is configured to: receive said each target and corresponding prediction as an input; receive a set of features associated with the input, the model, and triage-progressability of the input; wherein the triage-progressability relates to whether to progress the input for drug discovery; apply the set of features to the model to predict whether the input is triage-progressible; determine the triage-progressability score for the input based on said prediction; provide a second ranked list based on the triage-progressability determined for each target; and output the second ranked list from the model.

In a further aspect of the invention there is provided a method of training a machine learning model to evaluate a drug target candidate for drug discovery, the method comprising: providing a labelled data set comprising data describing a plurality of drug targets, where each drug target has been manually labelled with a binary label indicating a positive or negative human assessment of whether the drug target is deemed suitable for progression based on one or more criteria; determining a set of features associated with each drug target and the corresponding criteria; training the model to predict the binary label based on the set of features. Preferably the one or more criteria comprise a specified disease of interest. Preferably the one or more criteria comprise one or more of a safety threshold; a therapeutic evidence threshold; a novelty threshold; a biological mechanism of interest; a ligandibility threshold. Preferably the step of providing the labelled data set comprises receiving an input of the binary label through a user interface.

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a schematic diagram illustrating an example of predicting human-based annotations for drug discovery according to the invention;

FIG. 1b is a flow diagram illustrating another example of predicting human-based annotations for drug discovery according to the invention;

FIG. 2a is a schematic diagram illustrating an example of ranking drug targets based on triage-progressability of the drug targets for drug discovery according to the invention;

FIG. 2b is a flow diagram illustrating another example of ranking drug targets based on triage-progressability of the drug targets for drug discovery according to the invention;

FIG. 3a is a block diagram illustrating an example of an interface for providing human-based annotations according to the invention;

FIG. 3b is a block diagram illustrating an example of the human-based annotations according to the invention;

FIG. 4 is a schematic diagram of a computing device suitable for implementing embodiments of the invention.

Common reference numerals are used throughout the figures to indicate similar features.

### DETAILED DESCRIPTION

Embodiments of the present invention are described below by way of example only. These examples represent the suitable modes of putting the invention into practise that are currently known to the applicant, although they are not the only ways in which this could be achieved. The description sets forth the functions of the example and the sequence of steps for constructing and operating the example. However, the same or equivalent functions and sequences may be accomplished by different examples.

In typical Machine Learning (ML) models, annotation is the process of labelling data and is completed manually by a human. Annotation is imperative in setting the foundations for training the ML model with the necessary data the model needs to understand the desired results, and ultimately make accurate predictions. The annotations state whether target predictions matches or not according to a range of criteria, such as novelty, safety, ligandability or tissue expression. It is appreciated that other criteria may be used with a similar method for other aspects of drug discovery, such as compound selection or lead optimisation. In practice, drug discovery experts tend to make these annotations based on a broader context, drawing upon their background knowledge and experience. Taking human annotations or human-based annotations into consideration, SAMMI—an ML-based workflow that uses human annotations as labels for training models—learns to imitate this human behaviour in order to learn a proxy-model of how experts make those decisions (i.e. in designating targets as ‘matches’ or ‘not matches’, or equivalently “triage progressible” or “not triage progressible”). Deploying SAMMI in effect enhances the quality of predicted targets or improves predictions in drug discovery.

Herein disclosed the means for predicting and evaluating triage-progressability using human-based annotations. Human-based annotations refer to annotations provided by a user when using SAMMI. Human-based annotations are utilized in SAMMI or in the context of using SAMMI for drug discovery or making informed decisions on progressing a drug target candidate. SAMMI includes at least one method or system for predicting human-based annotations with respect to identifying potential drug target candidates from the input target. One or more ML models evaluate the input to SAMMI. The ML models are deployed to learn from human-based (or manual) annotations. The annotations represent whether a given target is likely to be progressible or non-progressible. The annotations may be, for example a binary value indicating that the human annotator evaluates the target is progressible or non-progressible (i.e. suitable for drug discovery or not suitable). A determination is made for whether to progress the input target. This determination is made based on a set of drug discovery metrics or features. This set of features include but are not limited to the user's disease of interest, a biological mechanism of interest, and the relative importance of safety and novelty associated with the drug target candidate. In accordance, SAMMI predicts for a new or unseen drug target the likelihood that a human would have annotated the target as progressible or non-progressable based on the set of features and with respect to the user-specified criteria.

An exemplary method or embodiment of SAMMI conferring increased predictive power over existing use of human-based annotations or human annotations by receiving a target input to a model or more specifically an ML model trained using human-annotated data, and receiving a set of features associated with the target input, model, and triage-progressability of the target input. The human-annotated data may be annotated and associated with triage-progressability of whether to progress the input for drug discovery. The association between the annotations and the triage-progressability may be directly or indirectly related in the sense that there may be a correlation or mapping associated with the one or more annotations and whether the input is progressible based on the annotations. SAMMI may apply the set of features to the model to predict whether the target input is progressible and optionally generate a likelihood indicator of human-based annotations in relation to the prediction. This provides the model with an output of the prediction and the likelihood indicator. Thus, the application of human-based annotations to the input progressability improves triage of input targets for decision-making during drug discovery when using SAMMI.

The applications of the human-based annotations or human annotations in this application are therefore imperative in setting the foundations for utilizing any ML models herein described. In this application, embodiments of SAMMI enable efficient training of these ML models with necessary data that the model requires to understand the desired results, and ultimately make accurate predictions or to optimize the decision-making process.

The decision-making process during drug discovery may be an iterative process that could be achieved by deploying one or more predictive ML models, ML-based models, ML techniques, ML architectures or ML algorithms together with or without the user or additional external input. This application collectively coins these models, techniques, architecture, and algorithms as ML models or systems. A range of ML models or systems may be used with or integrated into SAMMI. The types of ML model or system depend on which approach is optimal for a given dataset.

The data set may be annotated or labelled by a human or as human-annotated data. The data underlying the data set may include data points corresponding to various interested entities and may serve as potential inputs to SAMMI. These entities may be associated with one or more drug target candidates. The entities may also be associated with diseases, biological processes, pathways and potential therapeutic targets and the like. The data corresponding to the entities of interest may be extracted from various structured and unstructured data sources, and literature via natural language processing or other data mining techniques. For example, the entities of interest included as part of the data set may be in the fields of bioinformatics and drug discovery where there is a need for human style triage-progressability scoring to reduce the need for manual analysis of large sets of drug targets.

Human-annotated data may be supplied with respect to a set of user-supplied criteria, such as the user's disease of interest, a biological mechanism of interest, or the relative importance of safety and novelty. Alternatively stated, the annotation indicating whether the target is progressible is provided in the context of a number of criteria. For example a particular target may be annotated as progressible for a particular specified disease, given a particular specified safety threshold and a particular novelty threshold. These criteria are used as inputs to the model with the drug target when training the model to predict the annotation and are equally provided to the trained model as inputs when predicting an annotation for an unseen drug target. Human-annotated data may be associated with a particular drug target candidate or drug target, where these associations could be based on biological rationale, therapeutic evidence, ligandability, novelty, molecular weight, chemical opportunity, chemical strategy, therapeutic strategy, patentability and legal enforcement based on Freedom to Operate data, and safety data. Examples of drug target candidate or drug target may include proteins, DNA, RNA, and the like. Examples of chemical opportunity may include indications of competitive advantages over developing drugs for some targets compared to other targets. These indications may be provided or derived using various algorithms or suitable techniques that could be integrated or adapted to be used with SAMMI. Examples of a therapeutic strategy include the consideration of therapeutic modality. Such therapeutic modality may range from small molecular drug/compound to biologics. Types of biologics include protein or other protein-based macromolecules such as antibodies and recombinant or fusion proteins. Other types of therapeutic modality may include RNA-based drugs (via RNA interference) targeting mRNAs.

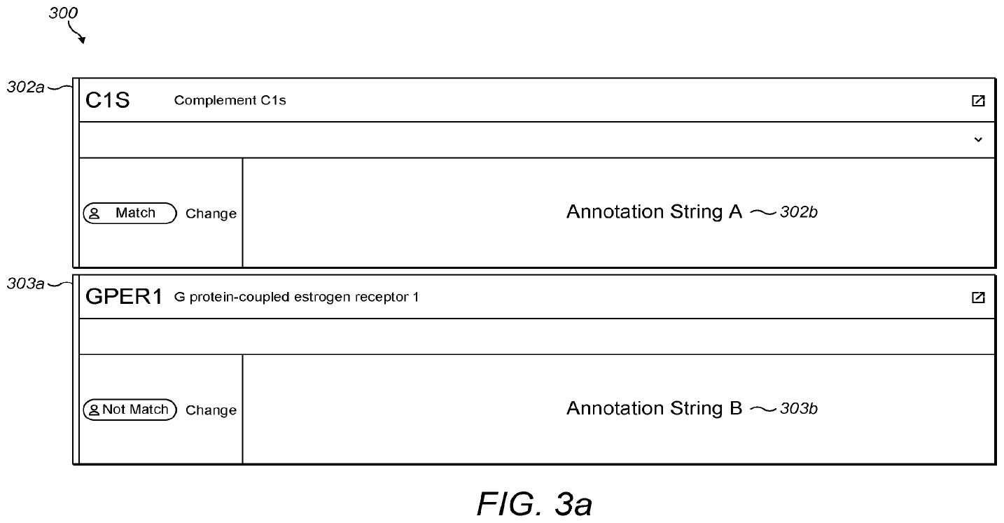

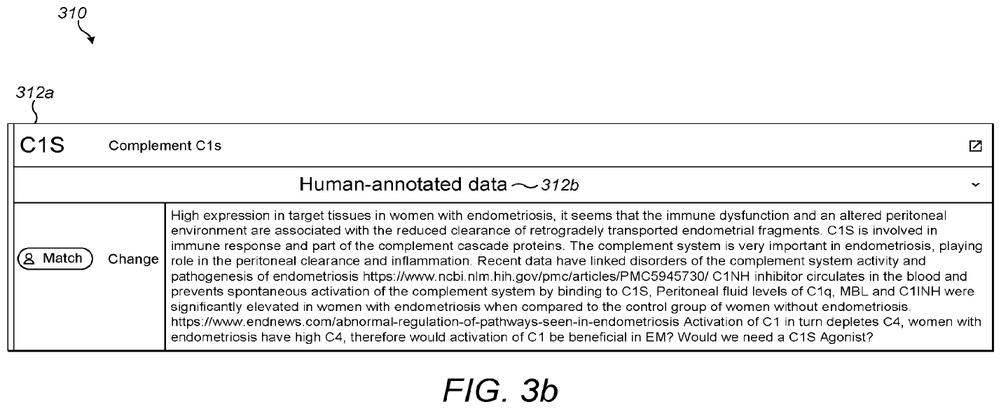

The ML models or systems used with SAMMI may include one or more trained ML algorithm or classifiers based on the data set referred to alternatively as training data associated with ‘known’ entities and/or entity types and/or relationships therebetween derived from large scale datasets (e.g. a corpus or set of text/documents or unstructured data). The training data may also include graph-based statistics associated with the fields of bioinformatics and drug discovery. Other fields may include but are not limited to, for example, chem(o)informatics, bioinformatics, and other informatics fields in relation to drug discovery, identification, and optimisation and other related products, treatment, analysis and/or modelling in these informatics fields. Thus, an ML model or system may correspond to a classifier or classification scheme that is generated using the training data as one or more models or systems used with SAMMI. Further detailed examples of the training data are described by subsequent text corresponding to FIG. 3a.

Examples of ML models or systems that may be used with SAMMI or in relation to this application may include by way of example only but are not limited to one or more of: any ML technique or algorithm/method that can be used to generate a trained model based on labelled and/or unlabelled training datasets; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, types of reinforcement learning, representation learning, similarity and metric learning, sparse dictionary learning, genetic algorithms, rule-based machine learning, learning classifier systems, and/or one or more combinations thereof and the like.

In the context of this application, once drug targets are identified based on biological relevancy, the likelihood of success consequently depends on whether the target is triage-progressible. Triage-progressability refers to whether the target may be used or selected for later stages in drug discovery, for example the target may be examined in vitro for binding affinity. Triage-progressability of a target is determined based on a set of triage-progressability criteria or features. These features can be derived from biological and non-biological rationale such as ligandability, safety, or therapeutic evidence. Triage-progressability could be determined by informative features both directly (i.e. because a human annotator observed the value of the feature in relation to the drug target and used the feature to determine triage-progressibility) or indirectly (i.e. a feature which the human annotator did not directly observe, but nonetheless can be used to predict their decision on triage-progressibility). An ML model or system may be applied in relation to these features. When applicable, the ML model or system results tend to improve consistency in the scoring process allowing SAMMI to provide consistent scores. The consistent scores help limit the amount of human triage to the best or most relevant targets and control the variability in human decision making by reducing manual analysis when tackling large data sets, i.e. drug targets data.

The set of features for triage-progressability may depend on the ML models used or used in relation to the input data or human-annotated data. The ML models can learn from the human-annotation in order to provide an accurate assessment of triage-progressability. The human-annotation may be representative of whether a given target is likely to be progressible or non-progressible. This assessment is based on various features corresponding to the level of novelty, the safety of the target, the ligandability of the target, or tissue expression data when used with the target and the like. It will be appreciated that other features may be incorporated together or in addition. The use of other features may be case-dependent, meaning a set of features are selected based on what data is queried by the user across targets, the context applied (e.g. the disease of interest and safety/ligandability/novelty criteria) and what ML model is deployed. The data queried may, for example, be a disease, biological mechanism, or data associated with the target. It will be appreciated that other features may be incorporated together or in addition.

Examples of other features may relate to, for example and more specifically, factors such as overlap between the predictions, top correlations of objects in a database or top processes corresponding to the target, correlation of the predictions with metadata associated with database objects, predictions derived from ligandable drug target families, percentage of processes or pathways found in the enrichment of gene data in a training model and in enriched lists of the plurality of predictions, overlap between pathway enrichment or process enrichment data between the entities, a summary of relationships associated with the predictions to objects in a database, reduction to practice statement of association between the predictions and a disease context, and connectivity associated with protein-protein interactions.

Any one or more of the above may correspond to the set of features in relation to human-annotated data for determining triage-progressability. Each or any combination of the above may be applied to capture relevant characteristics of a prediction for assessing whether the input or drug target candidate is progressible or non-progressible during the triaging process while taking into consideration the input target or drug target candidate's human-based annotation concerns associated with biological rationale, therapeutic evidence, ligandability, novelty, molecular weight, chemical opportunity, chemical strategy, therapeutic strategy, patentability and legal enforcement based on Freedom to Operate data, and safety and the like. It is appreciated that the set of features may traverse or be derived from multiple fields or domains of knowledge that includes but are not limited to the drug discovery or the regulatory/legal compliance thereof.

The domain of the present disclosure may be described, by way of example only but are not limited to, with respect to biomedical, biological, chem(o)informatics or bioinformatics entities, presented or stored in the form of knowledge graphs or other appropriate data structures, are to be appreciated by the skilled person that the details of the present disclosure are applicable as the application demands to any other type of entity, information, data informatics fields and the like. For example, the ML models or systems described above can be applied to any of any other type of entity, information, data informatics fields and the like insofar described in the present disclosure.

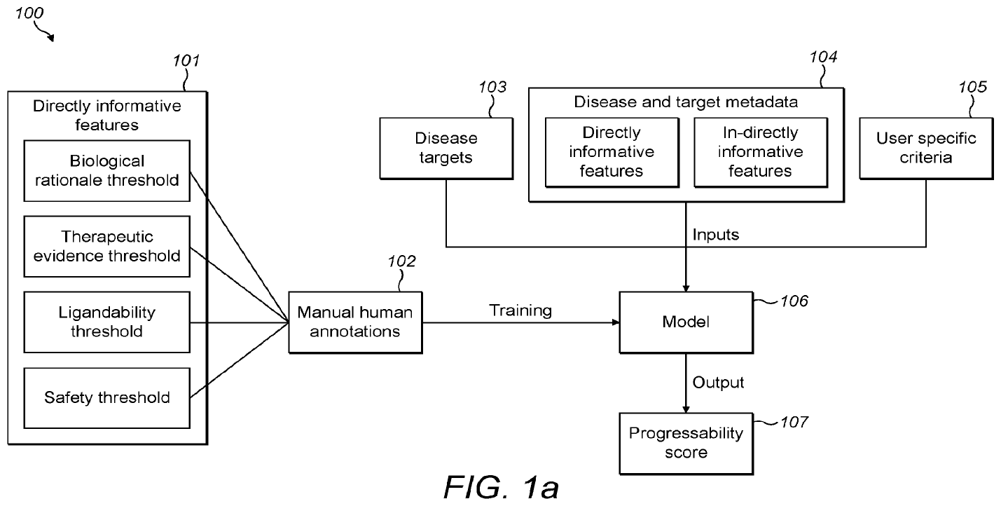

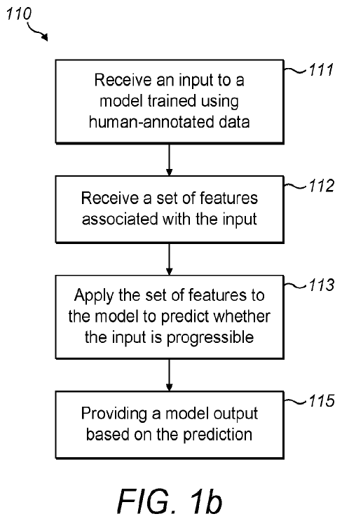

FIG. 1a is a schematic diagram illustrating an example workflow 100 of SAMMI. The workflow 100 is deployed to predict human-based annotations in the context of drug discovery. Here, SAMMI is shown to receive at least one input to the model 106 trained using human-annotated data. That is, the workflow 100 trains the model 106 based on manual human annotations 102. These manual human annotations 102 associated with the human-annotated data are created based on directly informative features 101, and derived, for example from data on the disease and target metadata, and informed by the annotators' background knowledge and experience thus producing a form of human-annotated data.

The directly informative features 101 could be applied independently to make triage-progressability assessments or as specific decisions prompted by a human annotator. Various criteria can serve as input features to the model and indicators of the triage strategy for making these assessments or decisions. The assessments or decisions may depend on various user specified criteria 105, such as those identified by the model's hyperparameters. They may include, for example biological rationale, therapeutic evidence, ligandability, safety, and the like.

The input may correspond to data associated with disease targets 103, disease and targets metadata 104, and user-specified criteria 105. The model 106 receiving the inputs may accept at least such data associated with disease target 103, disease and target metadata 104, including direct and indirectly informative features, and a user-supplied criteria 105. The model 106 is adapted to evaluate the triage-progressability of the drug targets 103 concerning the data. To reduce bias, the indirectly informative features within the disease and target metadata 104 could include features not seen by the human annotator, but that tends to predict some of the decisions made for the human annotations, such as the biological model performance scores that serve as independent evaluators.

On the other hand, user-supplied criteria 105 may include the user's disease of interest, the biological mechanism of interest or the relative importance of safety and novelty to the triage-progressability decision. These user-supplied criteria may be provided in the form of model performance scores. The scores may be differentiated statistically, for example, using confidence interval—selecting all targets with a score higher than 0.95.

The above enables the model in predicting the triage-progressability of the drug target based on the prior training, using the labels present in the manual annotations 102 or as human-annotated data for training, which comprise at least one informative features. The triage-progressability score 107 is provided in turn based on an imitation of the human-annotated data.

Arriving at the triage-progressability score 107, the workflow 100 of SAMMI potentially requires a set of features associated with at least the input, the model and the triage-progressability of the input. The set of features comprise at least one user-specified criteria 105 and a set of metadata 104 associated with the input, where the input may be a biological target and/or a target disease. The set of features is also received as input to the model 106 trained using manual human annotation 102 and used to compute the triage-progressability score 107. In accordance with FIG. 1b, the model applies the set of features to determine whether the input is progressabile and the model generates, as an option, a likelihood indicator of human-annotation based on the determination. The likelihood indicator may be computed as a part of the progressability score 107. The triage-progressability score 107, together with the likelihood indicator, may be outputted from the model 106 as the prediction for assessing triage-progressability.

The model output may be used for prioritizing targets, at least partly based on the likelihood indicator of human-annotation as an option, presented to human experts for triaging. Since conventional models could generate hundreds or thousands of “biologically relevant” targets for a single query potentially, the number of targets would no doubt take human experts too long to triage. To overcome this, the application of SAMMI helps narrow down or reduce the targets to a manageable subset of the top targets. The subset of the top targets would be considered most promising from the triage standpoint. The human experts would need only to review the subset of targets, thereby reducing the time required to perform the triage. SAMMI thus provides human experts with a more effective way of progressing a drug target candidate using human-based annotation.

FIG. 1b is a flow diagram illustrating another example workflow 110 of SAMMI in predicting human-based annotations for drug discovery. In this example, an input is received by a model of SAMMI trained using human-annotated data, together with the input drug target candidate and/or disease. SAMMI or the underlying model receives a set of features associated with the input, the model, and the triage-progressability of the input. The set of features is applied to the model to predict whether the input is progressible. As an option, a likelihood indicator of human-annotation or annotation associated with human-annotated data may be generated in relation to the prediction and outputted with the target.

In step 111, a model, trained using human-annotated data, is configured to receive an input. The input may be one or a combination of biological targets, along with a set of criteria including the disease of interest. The trained model is purposed for assessing whether the input is progressible. The model is trained using human-annotated data and the data is processed into the model parameters.

The human-annotated data used for training include at least one annotation associated with a triage-progressability of whether to progress the input for further drug discovery. The annotation may be provided manually as a numerical value or categorical text. For example, the numerical value could be 1 or 0, which corresponds to the exemplary text ‘MATCH’ or ‘NOT MATCH’. Each of the said at least one annotation thereby comprises an indicator for the triage-progressability.

The human-annotated data may also comprise positive training data and negative training data. The annotation may be done manually and provided as human-annotated training data. This data may be provided via or using an interface designed for an expert user. The human-annotated data comprise, or the annotation thereof may be associated with, at least one informative feature. The features may be a directly informative feature depending on the context and the ML models used. That is, the informative feature may be both shown to the user when they determine the annotation and used in training the mode.

In step 112, the model receives a set of features associated with a target. The set of features may also include at least one user-specified criteria and a set of metadata associated with the input. The metadata comprises directly informative features and indirectly informative features or collectively recognized herein as informative features. The informative features attribute to the determination of whether the input should be progressed.

The user-specified criteria may also be indicative of whether the input is progressible. At least one user-specified criteria comprise an indicator associated with the input. These indicators may be associated with a possible drug target candidate. That is, the association is based on at least one or more of: biological rationale, therapeutic evidence, ligandability, novelty, molecular weight, chemical opportunity, chemical strategy, therapeutic strategy, patentability and legal enforcement based on Freedom to Operate data, safety and the like.

In step 113, the model applies the set of features to predict or make the prediction of whether the input is progressible. The model may comprise one or more ML models or systems herein described. The models or systems may encompass and maintain at least one classifier trained using human-annotated training data. The model may be optimized in addition to using or employing techniques such as gradient boosting or logistic regression.

In step 114, a likelihood indicator of human-annotation may be optionally generated in relation to the prediction in step 113. The generated likelihood indicator of human-annotation provides a numerical representation of how likely the input is manually annotated. The likelihood indicator may serve as part of, or as an optional criterion to, the output for progressibility or for determining a progressibility score that is indicative of whether a target is a match or not.

In step 115, along with the prediction for progressability, the likelihood indicator in step 114 is also further considered, at least inherently as part of the model output. The prediction for progressability score is outputted as the prediction. The output is used to consolidate and prioritize a list of targets for triaging. That is, the consolidated and prioritized output from SAMMI provides human experts with the ability to handle a large number of targets initially produced from querying the predictive models or systems in relation to the SAMMI. These predictive models or system may be external or as an add-on to SAMMI.

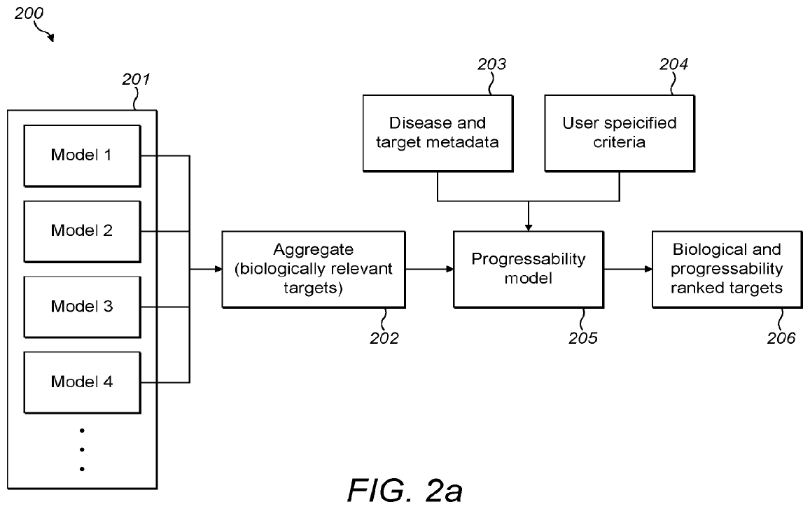

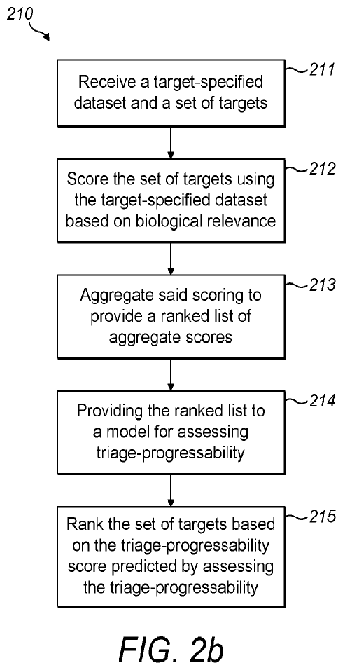

FIG. 2a is a schematic diagram illustrating yet another example workflow 200 of SAMMI. Here, the workflow 200 is deployed to rank drug targets based on triage-progressability of the drug targets. More than one or multiple models 201 may be used for input to the overall workflow 200. The workflow 200 may also include ranking and scoring of the drug targets based on triage-progressability criteria. Further input of manual analysis or human-annotated data may not be needed or in such case that the data is filtered before manual analysis.

The workflow 200, in particular, provides one or more scores pertaining to biological relevancy. The scores may be computed as the prediction made by the multiple models 201 and aggregated into a ranked list 202. This ranked list 202 of these biologically relevant targets and the disease target metadata 203 may serve as input to the progressability model 205 alongside the user-specified criteria 204, where the model 205 ranks the targets according to triage-progressability,

The input may also be in the form of a target-specified dataset, where the target-specified dataset comprises data representation of one or more of data derived from various literature sources and/or alternative databases aside from the set of multiple models 201. Starting with the input, the workflow 200 ranks the set of targets based on the target-specified dataset to generate a ranked list. As an option, the set of targets are scored using the target-specified dataset according to the targets' biological relevance providing the ranked list 202. The ranked list may be an aggregation of biologically relevant targets and the disease target metadata. The ranked list may include a list of targets and corresponding predictions ranked according to the scores.

The ranked list 202 is provided to the progressability model 205 according to the workflow 200. The progressability model 205 is configured to determine the triage-progressability scores for disease targets based on the triage-progressability criteria and the user-supplied criteria, and re-ranks the drug targets based on both the biological relevancy score and the triage-progressability score 206.

Specifically, the progressability model 205 may receive the targets from the ranked list and corresponding prediction from the multiple models 201 as model input. Together with the model input is a set of features. The set of features is associated with the input, the model, with triage-progressability of the input, where the triage-progressability refers to whether to progress the input for drug discovery based on triage-progressability criteria, the model 205 applies the set of features to predict whether the input is progressible based on the computed triage-progressability scores 206.

The set of features may be associated with the triage-progressability criteria or as metadata associated with the input, the user-supplied or specified criteria, and as such to produce triage-progressability scores. The triage-progressability scores 206 may depend on the likelihood of human-annotation. An indicator of the likelihood of human-annotation could be optionally determined by model 205 and used for the determination of the triage-progressability score. This may be done in relation to the prediction of whether to progress the targets from the initial ranked list 202. A second ranked list may be determined based on the triage-progressability and biological relevancy while taking into consideration the indicator for the likelihood of human-annotation. The second ranked list may in turn serve as model output comprising input targets to the multiple models 201, in a manner iteratively or establishing an iterative loop between the progressability model 205 and the multiple model 201. The second ranked list may be continuously updated.

The multiple models 201 receiving the input targets may comprise ML models or systems herein described. One or more aggregate models may replace the multiple models 201. The multiple models 201 or the model aggregate may be trained using annotated data and do not apply the same annotations associated with the multiple models 205 that is used for triage-progressiblity as opposed to the multiple models 201 focused on biological relevance. They may comprise one or more ML classifiers or classification schemes, such as those used with gradient boosted decision trees. They learn to classify a previously unseen input based on one or more pre-trained criteria that are biologically relevant.

In one specific example, the multiple models 201 or the aggregate model may be trained using 5-fold cross validation (CV) to select the hyperparameters. The best-performing hyperparameters may be selected during the CV search and used to further training of new models.

In general, the 201 models may be regarded as anything from knowledge graph inference models to network diffusion models; this could also include any arbitrary queries on our Natural Language Processing (NLP) processed data. The 201 models are not constrained to classification methods or gradient boosting methods as described above.

FIG. 2b is a flow diagram illustrating another example of ranking drug targets based on triage-progressability of the drug targets for drug discovery in accordance with the workflow of FIG. 2A. Prediction from a target-specified dataset may use as input to determining triage-progressability. Targets may be ranked to provide a ranked list. A score is determined as an option from each target using the target-specified dataset based on biological relevance. The scores are aggregated to provide this ranked list for assessing triage-progressability.

In step 211, the target-specified dataset and a set of targets are received by the progressability model. The target-specified dataset may comprise one or more of: a set of model, literature source, and alternative source of data. The target-specified dataset may be a data representation of the output or prediction from the set of models, data extracted from various literature sources, or from alternative sources of data stored in a gamut of databases.

In step 212, the set of targets are scored using the target-specified dataset based on biological relevance for ranking the targets. The biological relevance may be determined based on biological data and represented as a level of relevance corresponding to each target. A ranked list is in turn generated.

In step 213, the scores or predictions from step 212 may be aggregated to provide the ranked list of aggregate scores, where the ranked list comprises a list of targets and corresponding predictions ranked according to the scoring in step 212. Optionally, the prediction may also comprise a likelihood indicator. The likelihood indictor inherently may be part of the prediction of triage-progressability. The likelihood indicator is indicative of human-annotation during the decision-making process of whether target is progressible. The likelihood does not contemplate the actual annotation. Instead, it is computed based on the probability that something will be annotated. In other words, the likelihood indicator does not necessarily consider the underlying annotation or what the annotation is, but instead compute the likelihood that something would be annotated. In effect, the biological relevance predictions/rankings determine whether the targets are sent as inputs to SAMMI; they do not directly determine whether a human would annotate the target. Only indirectly, by way of association, the indicator helps assess whether the input will be human-annotated or derivable from human-annotated data.

In step 214, the ranked list is provided to the progressability model for further assessment. The progressability model predicts a triage-progressability score for each target in the ranked list.

In step 215, the set of targets are ranked or re-ranked based on the triage-progressability score predicted by assessing the triage-progressability. A second list of the targets may serve as the output to the progressability model. The second list may be a consolidated and prioritized list of targets used for triaging.

The progressability model may receive input of the features set comprising at least one user-specified criteria and a set of metadata associated with the input. The set of metadata may comprise at least one informative feature.

The progressability model may be trained using human-annotated data. The progressability model may receive a subset of human-annotated data, where the subset of human-annotated data is annotated for triage-progressability. That is, the human-annotated data comprises at least one annotation associated with the triage-progressability. The annotation may be certain categorical text or numerical values manually designated. The model is configured to identify a set of model features for the subset of human-annotated data such as those inputted to the model as the features set comprising at least one user-specified criteria and a set of metadata associated with the input. The model comprises one or more ML classifiers that help identify and select the subset of human-annotated data based on the set of model features. The model is updated. The updated model evaluates whether the set of human-annotated data is triage-progressible, where triage-progressability associated with a model output may be generated. The model is deployed for evaluation of whether a set of data is triage-progressible.

FIG. 3a is a block diagram illustrating an example of an interface 300 for providing human-based annotations. The interface 300 is configured to receive input data as strings, shown as annotated string A 302b and annotated string B 303b, where each string is associated with a target and evaluated using the progressability model.

According to the figure, the first target is C1S (Complement C1S) 302a and the second target is GPER1 (G-protein coupled estrogen receptor 1) 303b. These targets may be part of a list of targets or individually as targets to be evaluated by the progressability model. Each target may comprise its annotated string referring to the human-annotated data. The annotated strings, together with its annotation, may also serve as training data for training the progressability model. The training data may be human-based annotations corresponding to the label ‘MATCH’ or the label ‘NOT MATCH’ as shown in the figure. The labels designate whether annotations match or not match based on a manual assessment made by a human who is expert in the field of GPER1 or C1S.

Further input or data for training the progressability model may include target (T) level informative features for triage-progressability, user-specified or defined criteria (DE) for the model operation, for example covering deployment and triage strategy, disease-target (DT) level biological relevance data (acquired from the drug discovery workflow) and target and tissue (TiT) specific data that can affect triage-progressability scoring. These examples include but are not limited to the following features and their corresponding values:
(De) deployment_safety_importance: 2
(De) deployment_ligandability_importance: 3
(T) target_family: Kinase
(T) target_compound_library_num_tools: 7
(T) target_ppi_degree: 30
(T) target_pathway_num: 7
(T) target_num_reported_side_effects: 0
(DT) disease_target_KGE_model_score: 0.7
(DT) disease_target_connected_in_graph: False
(DT) disease_target_num_two_hop_paths_in_graph: 32
(DT) disease_target_num_sentence_cooccurence: 3
(DT) disease_target_clinical_trials: 0
(TiT) tissue_target_diff exp: False
(TiT) tissue_target_num_sentence_cooccurance: 3

Collectively, the numerical value associated with the above-listed features may serve as training data for the purpose of training the progressability model or any ML model or system used with SAMMI.

The human-based annotations may state whether target predictions are matching or not matching according to a range of criteria or indicators, such as novelty, safety, ligandability or tissue expression, some of which may be determined by a human annotator. It will be appreciated that other criteria may be used with a similar method for other aspects of drug discovery, such as compound selection or lead optimisation. The set of criteria may be associated with the drug target candidate. They may include but are not limited to at least one of biological rationale, therapeutic evidence, ligandability, novelty, molecular weight, chemical opportunity, chemical strategy, patentability and legal enforcement based on Freedom to Operate data, and safety.

During training, the human-annotated labels are represented with a binary ‘MATCH’ (1) or ‘NOT MATCH’ (0) input. The progressability model then learns a regression against these labels. During the evaluation, the model will generate a score indicating the predicted probability of the sample being a match. For example, an evaluation mode output above 0.5 would indicate a sample that would be more likely categorised by a human annotation as a ‘MATCH’ compared to a ‘NOT MATCH’.

FIG. 3b is a block diagram illustrating an example of the human-based annotations described according to FIG. 2a. In particular, FIG. 3b details an example of a text string corresponding to human-annotated data 312b comprise an annotation. The annotation is ‘MATCH’. The annotation suggests that the first target being C1S (complement C1S) 312a a match. The human-annotated data typically comprise any statement, question or textual data associated with the target. As shown in the figure, the text string corresponding to the human-annotated data details the biological context or mechanism associated with the first target C1S 312a to the extent suggests the biological relevance of the first target for scoring.

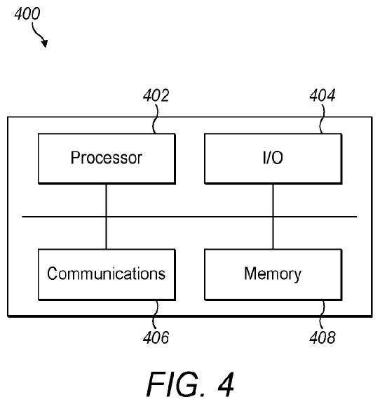

FIG. 4 is a schematic diagram illustrating an example computing apparatus/system 400 that may be used to implement one or more aspects of the system(s), apparatus, method(s), and/or process(es) combinations thereof, modifications thereof, and/or as described with reference to FIGS. 1 to 3 and/or as described herein. Computing apparatus/system 400 includes one or more processor unit(s) 401, an input/output unit 402, communications unit/interface 403, a memory unit 404 in which the one or more processor unit(s) 401 are connected to the input/output unit 402, communications unit/interface 403, and the memory unit 404. In some embodiments, the computing apparatus/system 400 may be a server, or one or more servers networked together. In some embodiments, the computing apparatus/system 400 may be a computer or supercomputer/processing facility or hardware/software suitable for processing or performing the one or more aspects of the system(s), apparatus, method(s), and/or process(es) combinations thereof, modifications thereof, and/or as described with reference to FIGS. 1 to 5 and/or as described herein. The communications interface 403 may connect the computing apparatus/system 400, via a communication network, with one or more services, devices, the server system(s), cloud-based platforms, systems for implementing subject-matter databases and/or knowledge graphs for implementing the invention as described herein. The memory unit 404 may store one or more program instructions, code or components such as, by way of example only but not limited to, an operating system and/or code/component(s) associated with the process(es)/method(s) as described with reference to FIGS. 1 to 3, additional data, applications, application firmware/software and/or further program instructions, code and/or components associated with implementing the functionality and/or one or more function(s) or functionality associated with one or more of the method(s) and/or process(es) of the device, service and/or server(s) hosting the process(es)/method(s)/system(s), apparatus, mechanisms and/or system(s)/platforms/architectures for implementing the invention as described herein, combinations thereof, modifications thereof, and/or as described with reference to at least one of the FIGS. 1 to 3.

In the embodiments, examples, and aspects of the invention as described above such as process(es), method(s), and/or system(s) of SAMMI may be implemented on and/or comprise one or more cloud platforms, one or more server(s) or computing system(s) or device(s). A server may comprise a single server or network of servers; the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

The above description discusses embodiments of the invention with reference to a single user for clarity. It will be understood that in practice, the system may be shared by a plurality of users, and possibly by a very large number of users simultaneously.

The embodiments described above may be configured to be semi-automatic and/or are configured to be fully automatic. In some examples a user or operator of the querying system(s)/process(es)/method(s) may manually instruct some steps of the process(es)/method(es) to be carried out.

The described embodiments of the invention a system, process(es), method(s) and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of predicting human-based annotations for drug discovery, the computer-implemented method comprising:
receiving an input to a model trained using human-annotated training data, wherein the human-annotated training data comprises at least one annotation associated with a triage-progressability annotation of whether to progress the input for the drug discovery;
receiving a set of features associated with the input, the model, and a triage-progressability of the input;
applying the set of features to the model to predict whether the input is triage-progressible; and
providing a model output based on the prediction.

2. The computer-implemented method of claim 1, further comprising:
generating a likelihood indicator of human-annotation in relation to the prediction; and providing the model output based on the likelihood indicator.

3. The computer-implemented method of claim 1, wherein the input comprises one or a combination of: a biological target and a target disease.

4. The computer-implemented method of claim 1, wherein the model output is used to consolidate and prioritize a list of targets for triaging.

5. The computer-implemented method of claim 1, wherein the human-annotated data comprises at least one informative feature.

6. The computer-implemented method of claim 1, wherein said at least one annotation comprises an indicator for the triage-progressability.

7. The computer-implemented method of claim 1, wherein the set of features comprise at least one user-specified criteria and a set of metadata associated with the input.

8. The computer-implemented method of claim 1, wherein the model comprises a machine learning (ML) classifier trained using human-annotated training data.

9. The computer-implemented method of claim 1, wherein the human-annotated training data comprises positive training data and negative training data.

10. The computer-implemented method of claim 7, wherein said at least one user-specified criteria comprises an indicator associated with the input.

11. The computer-implemented method of claim 10, wherein the indicator is associated with a drug target candidate, the association is based on at least one or more of: biological rationale, therapeutic evidence, ligandability, novelty, molecular weight, chemical opportunity, chemical strategy, therapeutic strategy, patentability and legal enforcement based on Freedom to Operate data, and safety.

12. The computer-implemented method of claim 1, wherein the human-annotated training data is provided via an interface.

13. A computer-implemented method for providing a model for predicting triage-progressability, the computer-implemented method comprising:
receiving a subset of human-annotated data, wherein the subset of human-annotated data is annotated for triage-progressability;
identifying a set of model features for the subset of human-annotated data;
classifying the subset of human-annotated data based on the set of model features; and
updating the model to evaluate whether the subset of human-annotated data is triage-progressible, wherein the model is configured to generate the triage-progressability associated with a model output, wherein the model is used for evaluation of whether a set of data is triage-progressible.

14. A computer-implemented method of claim 13, wherein the set of model features comprise informative features, and features associated with user-specified criteria.

15. A computer-implemented method of claim 13, further comprising:
optimizing the model in relation to the subset of human-annotated data.

16. A computer-implemented method of claim 13, wherein the subset of human-annotated data is selected from the set of human-annotated data based on one or more validation techniques.

17. A computer-implemented method for ranking drug targets based on triage-progressability of the drug targets, wherein the computer-implemented method comprising:
receiving a target-specified dataset and a set of drug targets, wherein the target-specified dataset comprises one or more of: a set of model, literature source, and alternative source of data;
scoring the set of targets using the target-specified dataset based on biological relevance;
aggregating said scoring to provide a ranked list of aggregate scores, wherein the ranked list comprises a list of drug targets and corresponding predictions ranked according to said scoring;
providing the ranked list to a model for predicting triage-progressability, wherein the model is configured to predict a triage-progressability score for each drug target of the ranked list; and
ranking the set of drug targets based on the triage-progressability score predicted by assessing the triage-progressability.

18. The computer-implemented method of claim 17, wherein the model is trained using human-annotated data, wherein the human-annotated data comprises at least one annotation associated with a triage-progressability annotation of whether to progress the set of drug targets for drug discovery.

19. A system for predicting human-based annotations for drug discovery, the system comprising:
an input module configured to receive an input to a model trained using human-annotated data, wherein the human-annotated data comprises at least one annotation associated with a triage-progressability of whether to progress the input for the drug discovery;
the input module is further configured to receive a set of features associated with the input, the model, and the triage-progressability of the input;
an evaluation module configured to apply the set of features to the model, predicting whether the input is progressible; and
an output module configured to provide a model output based on the prediction.

20. The system of claim 19, wherein the system is configured according to:
generate a likelihood indicator of human-annotation in relation to the prediction; and
provide the model output based on the likelihood indicator.

21. (canceled)

22. A system for drug discovery based on triage-progressability, the system comprising a processor and a memory storing instructions, which, when executed by the processor, cause the processor to:
receive a target-specified dataset and a set of targets, wherein the target-specified dataset comprises one or more of: a set of models, a collection of literature sources, and a compilation of data from external sources;
rank the set of targets based on the target-specified dataset to generate a ranked list;
provide the ranked list to a model for predicting the triage-progressability, wherein the model is configured to predict a triage-progressability score for each target of the ranked list; and for each target of the ranked list, the model is configured to:
receive said each target and corresponding prediction as an input;
receive a set of features associated with the input, the model, and triage-progressability of the input; wherein the triage-progressability relates to whether to progress the input for drug discovery;
apply the set of features to the model to predict whether the input is triage-progressible;
determine the triage-progressability score for the input based on said prediction;
provide a second ranked list based on the triage-progressability determined for each target; and
output the second ranked list from the model.

23. The system of claim 22, wherein the system is further configured to:
score the set of targets using the target-specified dataset based on biological relevance; and
aggregate said score to provide a ranked list of aggregate scores, wherein the ranked list comprises a list of targets and corresponding predictions ranked according to said score.

24. The system of claim 22, wherein said prediction comprises an indicator of likelihood that the input is annotated by human.

25. The system of claim 22, wherein the system is further configured to:
generate a likelihood indicator of human-annotation in relation to the prediction; and
provide the model output based on the likelihood indicator.
